(19) United States
(12) Patent Application Publication
(10) Pub. No.: US 2007/0231414 A1
(43) Pub. Date: Oct. 4, 2007

(54) COMPOSITION HAVING BONE RESORPTION INHIBITION-RELATED EFFECT AND METHOD FOR INHIBITING BONE RESORPTION

(75) Inventors: Fumiki Aoki, Hyogo (JP); Yuji Tominaga, Hyogo (JP); Takayuki Sakogawa, Hyogo (JP); Shinichi Yokota, Hyogo (JP); Seiki Wada, Saitama (JP)

Correspondence Address: CONNOLLY BOVE LODGE & HUTZ LLP, 1875 EYE STREET, N.W., SUITE 1100, WASHINGTON, DC, 20036, US

(73) Assignee: KANEKA CORPORATION, Osaka (JP)

(21) Appl. No.: 11/692,288

(22) Filed: Mar. 28, 2007

(30) Foreign Application Priority Data
Mar. 29, 2006 (JP) 2006-091489

(51) Int. Cl. A61K 36/82; A61K 36/53; A61K 36/28

(52) U.S. Cl. 424/725; 424/729; 424/745; 424/764

(57) ABSTRACT

The present invention has for its object to provide a method which comprises using edible and safe food components as active ingredients and promotes the OPG production promoting effect, RANKL expression inhibiting effect and osteoclast differentiation inhibiting effect, which take part in the prevention of bone resorption, and a composition like that. The present invention provides a method for promoting the bone resorption inhibitory effect such as the OPG production promoting effect, RANKL expression inhibiting effect and osteoclast differentiation inhibiting effect by using a composition containing extracts from plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and/or plants belonging to the genus Ptychopetalum, and a composition like that.

### TECHNICAL FIELD

The present invention relates to a composition having a bone resorption inhibition-related effect, in particular an osteoprotegerin (OPG) production promoting effect, a receptor activator of NF-κB ligand (RANKL) expression inhibiting effect or an osteoclast differentiation inhibiting effect, and a method for expressing said effect by administering these composition.

### BACKGROUND ART

Mammalian bones are always subject to repeated bone resorption and bone formation (osteogenesis). The cells involved in osteogenesis are osteoblasts and the cells involved in bone resorption are osteoclasts. The growth, maintenance and repair of bones depend on the rate balance between the formation of these cells and the resorption. Once that balance is disturbed, bone resorption outruns osteogenesis, whereby bone metabolism-related diseases resulting from decreases in bone quantity, for example osteoporosis, are caused. For the maintenance and/or enhancement of the bone quantity, it is therefore important that osteogenesis should not fall below bone resorption (Non-Patent Document 1).

Osteoporosis is a systemic bone disease causing bone quantity decreases and osseous tissue degeneration as a result of decreases in calcium and collagen, which form bones. Osteoporosis is frequently found in aged women, in particular postmenopausal women, and the frequency thereof has been increasing with the lengthening life spans in recent years. Bone embrittlement due to osteoporosis induces pain and/or an increased level of risk of fracture causing bedriddenness, thus becoming a problem capable of greatly influencing the quality of life of elderly persons (Non-Patent Document 2).

Osteoclasts are derived from undifferentiated hematopoietic cells through multinucleation and differentiation, which give mature osteoclasts functioning in bone resorption. It is known that osteoclast differentiation is promoted by a receptor activator of NF-κB ligand (hereinafter referred to as RANKL) expressed in osteoblasts. On the other hand, it is known that an osteoprotegerin (hereinafter referred to as OPG) is also secreted from osteoblasts and binds to RANKL to inhibit the osteoclast differentiation promoting effect of RANKL (Non-Patent Document 3). In view of the foregoing, inhibition of the expression of RANKL and promotion of the secretion of OPG in osteoblasts can be expected to be effective in inhibiting osteoclast differentiation and thereby inhibiting bone resorption. Further, compounds capable of directly inhibiting osteoclast differentiation without inhibiting RANKL expression or promoting OPG production in osteoblasts have been discovered, and such compounds can also be expected to inhibit bone resorption (Non-Patent Document 4).

As regards OPG, in particular, various administration tests have been made and it has been established that OPG is effective not only against osteoporosis due to menopause, immobilization, use of steroidal preparations or use of immunosuppressants but also against rheumatoid arthritis, periodontitis and so forth (Non-Patent Document 5). As mentioned above, OPG is possibly effective against various diseases. Since, however, OPG itself is a protein, a problem arises: the possibility of its being absorbed as such upon oral administration is low.

Therefore, various components showing OPG production promoting activity in viva have been searched for to obtain the effects of OPG via oral administration. For example, Jude et al. have reported that the administration of low-molecular compounds capable of promoting OPG production to disease model rats with osteoporosis or rheumatism is effective in the treatment or prevention of these diseases (Non-Patent Document 6). Further, among food components, some have been found to have OPG production promoting activity and they are very useful from the safety and convenience viewpoint. As such food components, there may be mentioned lactoferrin, trehalose and the like. Among them, lactoferrin promotes OPG production not only from osteoblasts but also from intestinal tract-derived cells, hence has better effects (Patent Document 1). However, it is a problem with lactoferrin that it is expensive since the raw material therefor is milk.

Chinese bellflower has been considered effective in the treatment of coughing and phlegm and has long been used as a galenical. In Korea, it is used for food in the form of kimchee. The use of hot water extracts of Chinese bellflower for bone growth has been proposed because of their ability to promote the production of IGF-1, which is a growth factor produced mainly in the liver (Patent Document 2).

Oregano is a plant widely used for food as a spice. Components extracted from leaves thereof with such an organic solvent as aqueous ethanol, ethanol or hexane have antimicrobial activity and therefore are used as food additives for keeping foods for a longer time (Non-Patent Document 7).

Thyme is a plant used for food as a spice; it has antimicrobial activity and, in addition, is said to be effective against anemia, coughing and sore throats, among others. It has been reported that thyme has a bone resorption inhibiting effect (Non-Patent Document 8).

Wild thyme is a herb of the same genus as thyme and is used as a food in the same manner.

Japanese pepper is a spice widely used for food in Japan and is known to have stomachic, cordial, anthelminthic and other effects. Japanese pepper is also known to promote collagen production when used in admixture with a plant belonging to the genus Pfaffia, a plant belonging to the genus Ajuga, and a plant belonging to the genus Rhaponticum, and the use thereof for such purpose has been proposed (Patent Document 3). In addition, the use thereof related to its action as an activator for aromatase involved in estrogen biosynthesis (Patent Document 4) and to bone resorption inhibition has been proposed.

Sichuan pepper is a spice widely used for food in China.

Seeds of avocado are widely used for food and, since tocotrienol and proanthocyanidin contained in such plants as avocado are components having bone resorption inhibiting activity, bone resorption inhibiting compositions derived from avocado have been proposed. Further, unsaponifiable components of avocado, in particular furan lipids, are effective against arthritis and the use thereof for osteoporosis has been proposed (Patent Document 5 to 7).

Seeds of anise have long been used in the form of herb tea and in giving dishes a fragrance.

Stevia contains a terpenoid glycoside called stevioside, which is a sweetening component, and therefore now is a food additive to be used as a sweetening agent.

Mate is a plant of Brazilian origin and is drinkable in the form of mate tea. Mate contains a small amount of caffeine and a large amount of mateine, which is a kind of alkaloid.

In Japan, tea has long been used in the form of drinks. Drinks are prepared by decocting raw or dried leaves, processed materials derived therefrom by heating and crumpling procedures, or semi-fermented or fully fermented products derived from raw leaves. As species thereof, there may be mentioned green tea, oolong tea and black tea, among others. Tea has bone resorption inhibiting activity and the use thereof for such activity has been proposed (Patent Document 8).

Herb teas prepared by decocting blue mallow are drunk with pleasure since the herb teas change color with the lapse of time.

Burdock is widely used for food.

Muira puama is used for food as a medicinal herb in Brazil.

Chamomile is used for food mainly in the form of herb tea and is said to be effective against insomnia and so forth.

Patent Document 1: Japanese Kokai Publication 2004-115509

Patent Document 2: Japanese Kokai Publication 2004-518647

Patent Document 3: Japanese Kokai Publication 2005-255527

Patent Document 4: Japanese Kokai Publication 2005-343872

Patent Document 5: Japanese Kohyo Publication 2002-520280

Patent Document 6: Japanese Kokai Publication 2004-262818

Patent Document 7: Japanese Kohyo Publication 2003-509506

Patent Document 8: Japanese Kokai Publication Hei-06-183985

Non-Patent Document 1: Nippon Rinsho (Japanese Journal of Clinical Medicine), 2002, Vol. 60, Extra Issue No. 3, pp. 34-37

Non-Patent Document 2: Igaku no Ayumi (Journal of Clinical and Experimental Medicine), 2001, Vol. 198, No. 9, pp. 574-579

Non-Patent Document 3: Endocrine Reviews, 1999, No. 20, Vol. 3, pp. 345-357

Non-Patent Document 4: Biological & Pharmaceutical Bulletin, 2004, Vol. 24, No. 4, pp. 504-509

Non-Patent Document 5: Nippon Rinsho, 2002, Vol. 60, Extra Issue No. 3, pp. 679-687

Non-Patent Document 6: The Journal of Pharmacology and Experimental Therapeutics, Vol. 309, No. 1, pp. 369-379

Non-Patent Document 7: Commentary on the List of Items Contained in the Inventory of Existing Additives, page 126, 1999, Published by the Japan Food Additives Association

Non-Patent Document 8: Bone, 2000, Vol. 32, pp. 372-380

### SUMMARY OF THE INVENTION

Among such edible plants as mentioned above, some have been suggested or proposed for their involvement in bone resorption inhibition and/or bone diseases. However, the mechanisms possibly involved have scarcely been elucidated and, for most of them, no evidence has been given as to the actual efficacy thereof. In addition, for a number of plant species, no suggestion has been given at all about their possible bone resorption inhibiting activity or their possible effect on bone diseases.

### DETAILED DESCRIPTION OF THE INVENTION

The present invention has for its object to provide a method which comprises using edible and safe food components as active ingredients and promotes the OPG production promoting effect, RANKL expression inhibiting effect and osteoclast differentiation inhibiting effect, which take part in the prevention of bone resorption, and a composition like that.

The present inventors found that organic solvent extracts from plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum have such an effect useful in bone resorption inhibition as an OPG production promoting effect, a RANKL expression inhibiting effect or an osteoclast differentiation inhibiting effect. Such finding has now led to completion of the present invention.

It has not been known at all that organic solvent extracts from the plants mentioned above have such an effect as mentioned above.

Thus, the present invention provides the following.

A method for promoting production of OPG, which comprises administrating to a subject a composition containing, as an active ingredient, organic solvent extracts from at least one plant species selected from the group consisting of plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum.

A method for inhibiting differentiation of osteoclast, which comprises administrating to a subject a composition containing, as an active ingredient, organic solvent extracts from at least one plant species selected from the group consisting of plants belonging to the genus Platycodon, plants belonging to the genus Persea, plants belonging to the genus Stevia, plants belonging to the genus Pimpinella, plants belonging to the genus Matricaria, and plants belonging to the genus Malva.

A method for inhibiting expression of RANKL, which comprises administrating to a subject a composition containing, as an active ingredient, organic solvent extracts from at least one plant species selected from the group consisting of plants belonging to the genus Platycodon, plants belonging to the genus Origanum, plants belonging to the genus Thymus, plants belonging to the genus Persea, plants belonging to the genus Pimpinella, and plants belonging to the genus Ilex.

In addition, the present invention provides the following.

A method for alleviating or preventing a disease associated with at least one action of the group consisting of OPG, RANKL and osteoclast differentiation inhibition, which comprises administrating to a subject a composition containing, as an active ingredient, organic solvent extracts from at least one plant species selected from the group consisting of plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum.

The method mentioned above, wherein the disease associated with at least one action of the group consisting of OPG, RANKL and osteoclast differentiation inhibition is osteoporosis.

Furthermore, the present invention provides the following.

The method mentioned above, wherein the composition is for eating or drinking.

The method mentioned above, wherein the composition is a medicine.

The method mentioned above, wherein the subject is a mammal.

The method mentioned above, wherein the subject is a human.

And then, the present invention provides the following.

An OPG production promoter which contains, as an active ingredient, an organic solvent extract from at least one plant species selected from the group consisting of plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum.

An osteoclast differentiation inhibitor which contains, as an active ingredient, an organic solvent extract from at least one plant species selected from the group consisting of plants belonging to the genus Platycodon, plants belonging to the genus Persea, plants belonging to the genus Stevia, plants belonging to the genus Pimpinella, plants belonging to the genus Matricaria, and plants belonging to the genus Malva.

A RANKL expression inhibitor which contains, as an active ingredient, an organic solvent extract from at least one plant species selected from plants belonging to the genus Platycodon, plants belonging to the genus Origanum, plants belonging to the genus Thymus, plants belonging to the genus Persea, plants belonging to the genus Pimpinella, and plants belonging to the genus Ilex.

The composition of the invention which has a bone resorption inhibition-related effect is a composition containing, as an active ingredient, an organic solvent extract from a specific plant or plants. Specifically, it includes an OPG production promoter containing, as an active ingredient, an organic solvent extract from at least one plant species selected from the group consisting of plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum; an osteoclast differentiation inhibitor containing, as an active ingredient, an organic solvent extract from at least one plant species selected from the group consisting of plants belonging to the genus Platycodon, plants belonging to the genus Persea, plants belonging to the genus Stevia, plants belonging to the genus Pimpinella, plants belonging to the genus Matricaria, and plants belonging to the genus Malva; RANKL expression inhibitor containing, as an active ingredient, an organic solvent extract from at least one plant species selected from the group consisting plants belonging to the genus Platycodon, plants belonging to the genus Origanum, plants belonging to the genus Thymus, plants belonging to the genus Persea, plants belonging to the genus Pimpinella, and plants belonging to the genus Ilex; and compositions containing them.

As the plants belonging to the genus Platycodon, which may be used in the practice of the invention, there may be preferably mentioned Chinese bellflower, more preferably Platycodon grandiflorum, and the like. The part or region thereof is preferably the root which is edible, although the whole plant may also be used. Generally, saponins and polyphenols are abundantly contained in plants belonging to the genus Platycodon.

As the plants belonging to the genus Origanum, which may be used in the practice of the invention, there may be preferably mentioned oregano, more preferably Origanum vulgare, and the like. The part or region is preferably the foliar part which serves as the raw material for food additives, although the whole plant may also be used. Generally, plants belonging to the genus Origanum are abundant in such components as thymol and carvacrol.

As the plants belonging to the genus Thymus, which may be used in the practice of the invention, there may be preferably mentioned thyme and Wild thyme, more preferably Thymus Species and Thymus serpyllum, and the like. The part or region is preferably the terrestrial part, although the whole plant may also be used. Generally, thymol, borneol, linalool, carvacrol, polyphenols and saponins occur abundantly in plants belonging to the genus Thymus.

As the plants belonging to the genus Zanthoxylum, which may be used in the practice of the invention, there may be preferably mentioned Sichuan pepper and Japanese pepper, more preferably Zanthoxylum bungeanum and Zanthoxylum piperitum, and the like. The part or region is preferably the seeds which are widely used for food, although the whole plant may also be used. The plants belonging to the genus Zanthoxylum contain such pungent components as sanshool and sanshoamide, such fragrant essential oils as geraniol, as well as dipentene, citral and so forth.

As the plants belonging to the genus Arctium, which may be used in the practice of the invention, there may be preferably mentioned burdock, more preferably Arctium lappa, and the like. The part or region is preferably the root which is widely used for food.

As the plants belonging to the genus Camellia, which may be used in the practice of the invention, there may be preferably mentioned tea, more preferably Camellia sinensis, and the like. The part or region is preferably the foliar part which is widely utilized, although the whole plant may also be used. The foliar part is not restricted in its form but includes a raw or dried matter, a processed matter obtained therefrom through heating and crumpling procedures, and a product obtained by semi-fermentation or complete fermentation of raw leaves.

As the plants belonging to the genus Persea, which may be used in the practice of the invention, there may be preferably mentioned avocado, more preferably Persea americana, and the like. The part or region is preferably the fruits, although the whole plant may also be used. Preferably used is the sarcocarp, which is edible.

As the plants belonging to the genus Stevia, which may be used in the practice of the invention, there may be preferably mentioned stevia, more preferably Stevia rebaudiana, and the like. The part or region is preferably the foliar part which is used as the food additive raw material, although the whole plant may also be used. It is known that plants belonging to the genus Stevia contain a sweet component called stevioside and flavonoids.

As the plants belonging to the genus Pimpinella, which may be used in the practice of the invention, there may be preferably mentioned anise, more preferably Pimpinella anisum, and the like. The part or region is preferably the fruits which are edible, although the whole plant may also be used. It is known that plants belonging to the genus Pimpinella contain such components as anethole, cavicol, anisaldehyde, anisic acid, terpenes and coumarin.

As the plants belonging to the genus Matricaria, which may be used in the practice of the invention, there may be preferably mentioned chamomile, more preferably Matricaria recutita, and the like. The part or region is preferably the floral part, although the whole plant may also be used. Mainly, azulene, bisabolol, farnesene, flavonoids, coumarin and the like are contained in plants belonging to the genus Matricaria.

As the plants belonging to the genus Malva, which may be used in the practice of the invention, there may be preferably mentioned blue mallow, more preferably Malva sylvestris, and the like. The part or region is preferably the floral part, although the whole plant may also be used.

As the plants belonging to the genus Ilex, which may be used in the practice of the invention, there may be preferably mentioned mate, more preferably Ilex paraguariensis, and the like. The part or region is preferably the foliar part which can be decocted for drinking with ease, although the whole plant may also be used. Mainly, caffeine, theobromine, theophylline, saponins, chlorogenic acid and the like are contained in plants belonging to the genus Ilex.

As the plants belonging to the genus Ptychopetalum, which may be used in the practice of the invention, there may be preferably mentioned muira puama, more preferably Ptychopetalum olacoides, and the like. The part or region is preferably those parts which can be used as foods, except for the root, more preferably the bark, although the whole plant may also be used.

In the practice of the invention, the method of obtaining the extracts of the above-mentioned plants is not particularly restricted provided that the extracts are organic solvent extracts and that the extracts can produce a bone resorption inhibition-related effect. Herein, the organic solvent extracts refer to extracts obtained by extraction with an organic solvent-based solvent.

In terms of efficient extraction, the organic solvent to be used for such extraction is preferably an amphiphilic organic solvent. The amphiphilic organic solvent refer to the organic solvent miscible with both of hydrophilic solvents and hydrophobic solvents. The organic solvent includes, for example, alcohols such as methanol, ethanol, propanol, butanol, propylene glycol and glycerol; esters such as ethyl acetate; ketones such as acetone; aliphatic hydrocarbons such as heptane and hexane. Fats or oils such as cooking oils may also be used. Among them, the amphiphilic organic solvent includes alcohols such as methanol, ethanol, propanol, butanol, propylene glycol and glycerol. The solvents mentioned above may be used singly or mixtures of two or more of them may be used. The organic solvents mentioned above may also be used in the form of solvents mixed with water provided that the major component of the mixed solvent is an organic solvent, more specifically that the organic solvent content in the extractant solvent is not less than half. Ethanol or aqueous ethanol which can be used in the field of foods and can be removed with ease is preferably used.

Both extracts as obtained by extraction and extracts obtained after solvent removal therefrom are referred to herein as extracts.

The method for obtaining those organic solvent extracts from various plants which are active components in the composition according to the invention is not particularly restricted, and those organic solvent extracts can be obtained by the following solvent extraction method, for example. The extracts can be obtained by bringing the plant species mentioned above into contact with any of the solvents mentioned above in an amount of about 1 to 20 times, preferably about 1 to 10 times, the plant species, for example by immersing, and stirring the mixture or allowing the same to stand, followed by filtration, centrifugation or the like. Then, if necessary, the extracts obtained may be dried by such a method as concentration under reduced pressure, lyophilization or spray drying.

The materials to be brought into contact with the solvent may be in a raw or dried condition; from the storage viewpoint, however, the dried condition is preferred. The materials mentioned above may be used in the form as harvested, ground, cut, or powdered. The temperature for contacting with solvent is generally about 0 to 130° C., preferably about 1 to 80° C., and the extracts so referred to herein can appropriately be obtained at room temperature to a slightly elevated temperature, namely about 20 to 60° C. The period of contacting with the solvent is generally about 0.1 hour to 1 month, preferably about 0.5 hour to 7 days.

The extracts so referred to herein, if they are free of substances inappropriate for use as drinks, foods or medicines, can be used in the form of extracts as originally obtained or in the form of purified or semi-purified extracts. For the purification, use may be made of various chromatography techniques, for example the affinity chromatography purification method, or various filtration, precipitation or centrifugation techniques, for instance.

If desirable, the components in the extracts may respectively be in the form of a salt, ester or glycoside with a substance acceptable for drinking, eating, or pharmaceutical uses.

The bone resorption-related effects so referred to herein refer to OPG production promotion, RANKL expression inhibition and osteoclast differentiation inhibition. These effects are associated with the inhibition of differentiation of osteoclasts, which are bone-resorbing cells, and therefore are useful in inhibiting in vivo bone resorption and, accordingly, in alleviating or preventing diseases resulting from decreases in bone quantity, typically osteoporosis. The composition provided herein can act on osteoblasts or precursors of osteoclasts and inhibit in vivo bone resorption through osteoclast differentiation inhibition via promotion of OPG production from osteoblasts or inhibition of RANKL expression in osteoblasts or through osteoclast differentiation inhibition not mediated by such action.

While the OPG production promoting activity can be assessed by directly administering a substance to be evaluated to animals, it can also be estimated using osteoblasts. As the method of evaluation using cells, there may be mentioned, among others, the method comprising assaying OPG using cells or the culture supernatant and the method comprising assaying the messenger RNA for OPG. Specifically, OPG can be assayed by the enzyme immunoassay (ELISA) or western blotting method, for instance. As the messenger RNA assay method, there may be mentioned the northern blotting, RT-PCR, and DNA array methods, among others. When the activity of the sample shows a higher value as generally compared with the solvent control in at least one of these assays, the sample is evaluated as “having OPG production promoting activity”.

While the RANKL expression inhibiting activity can be assessed by directly administering a substance to be evaluated to animals, it can also be estimated using osteoblasts. As the method of evaluation using cells, there may be mentioned, among others, the method comprising assaying RANKL expressed in cells and the method comprising assaying the messenger RNA for RANKL. Specifically, RANKL can be assayed by the enzyme immunoassay (ELISA) or western blotting method, for instance. As the messenger RNA assay method, there may be mentioned the northern blotting, RT-PCR, and DNA array methods, among others. When the activity of the sample shows a lower value as generally compared with the solvent control in at least one of these assays, the sample is evaluated as “having RANKL expression inhibiting activity”.

While the osteoclast differentiation inhibiting activity can be assessed by directly administering a substance to be evaluated to animals, it can also be estimated using such cells as bone marrow cells, splenocytes ormacrophages. As the method of evaluation using such cells, there may be mentioned the method of evaluating the osteoclast differentiation inhibiting ability which uses a medium supplemented with RANKL and/or M-CSF, which induces osteoclast differentiation, and the method comprising cocultivating with osteoblasts using a medium supplemented with prostaglandin E2, adrenocortical hormone, interleukin-1β, active form vitamin D3, a lipopolysaccharide, or a like osteoclast differentiation stimulating reagent and evaluating the osteoclast differentiation inhibiting ability. In these cultivation methods, the osteoclast differentiation can be assessed in terms of expression of tartrate-resistant acid phosphatase (TRAP) which is specifically expressed in osteoclasts. More specifically, there may be mentioned, among others, the method comprising counting osteoclasts by the staining technique using a substrate capable of being caused to develop a color by TRAP or the calorimetric method using a substrate capable of being caused to develop a color by TRAP. When the activity of the sample shows a lower value as generally compared with the solvent control in at least one of these assays, the sample is evaluated as “having osteoclast differentiation inhibiting activity”.

The content of the organic solvent extract from the plants mentioned above in the composition of the invention is not particularly restricted provided that the content of the extract is within the range within which a bone resorption inhibition-related effect can be produced. The content of the extract is, for example, preferably 0.01 to 100% by weight, more preferably 0.1 to 100% by weight, relative to 100% by weight of the composition.

In short, the composition of the invention may contain various additives to be mentioned below and the like in addition to the extracts mentioned above, if necessary.

The composition for eating or drinking according to the invention is a composition containing the above-mentioned OPG production promoter, RANKL expression inhibitor or osteoclast differentiation inhibitor and is a mixture of a general food and such promoter or inhibitor. It may take the formof capsules, tablets, granules or like easily eatable or drinkable preparations as prepared by using a known carrier(s) and/or auxiliary agent(s) suited for eating or drinking. The composition for eating or drinking, so referred to herein, includes, for example, general foods, functional health foods (specific health foods, functional nutritive foods), health foods and nutritional supplements, among others. The general food, so referred to herein, includes, but is not limited to, drinks, dairy products, fermented milk products, lactic acid beverages, processed milk products, coffee-flavored drinks, juices, ice creams, candies, biscuits, wafers, jellies, soups and noodles. Preferred among them are drinks, dairy products, fermented milk products, lactic acid beverages, wafers and jellies.

The pharmaceutical composition of the invention is an OPG production promoter-containing, RANKL expression inhibitor-containing or osteoclast differentiation inhibitor-containing composition. Further, it may be the promoter or inhibitor itself and, if desired, it may be a composition further containing one or more pharmaceutically acceptable carrier. The use thereof is not restricted but mention may be made of drugs easily available such as over-the-counter (OTC) drugs, and quasi drugs, for instance. The form of the pharmaceutical composition is not restricted but includes, among others, pills, solutions, powders, granules, tablets, capsules, troches, syrups, dry syrups, elixirs, cachets and suppositories. Preferred are capsules, tablets, solutions, elixirs, cachets and suppositories, among others. The pharmaceutically acceptable carrier is any material suited for enabling oral, enteric, transdermal or subcutaneous administration and includes, among others, water, gelatin, gum arabic, lactose, microcrystalline cellulose, starch, sodium starchglycolate, calcium hydrogen phosphate, magnesium stearate, talc, colloidal silicon dioxide and the like.

The above-mentioned composition of the invention may also be a composition further containing at least one food or food additive said to be effective against such diseases as osteoporosis and rheumatism in association with OPG production promotion, RANKL expression inhibition or osteoclast differentiation inhibition, including, for example, extracts from soybean, pomegranate and the like, calcium, magnesium, vitamin D, vitamin K, glucosamine, chondroitin and collagen, and the like. The form thereof is not particularly restricted but includes compositions for drinking or eating and pharmaceutical compositions.

The composition of the invention can alleviate or prevent a disease associated with at least one action of the group consisting of OPG, RANKL and osteoclast differentiation inhibition.

The term “disease associated with at least one such action of the group consisting of OPG, RANKL and osteoclast differentiation inhibition” as used herein means any of those diseases the symptom(s) of which can be alleviated as a result of in vivo OPG production promotion, RANKL expression inhibition and/or osteoclast differentiation inhibition. More specifically, mention may be made of osteoporosis due to menopause, immobilization, use of steroid preparations and use of immunosuppressants and, further, rheumatoid arthritis, alveolar abscess and periodontitis, among others.

The method for promoting production of OPG, the method for inhibiting differentiation of osteoclast, and the method for inhibiting expression of RANKL, according to the invention, comprises administrating to a subject a composition containing, as an active ingredient, organic solvent extracts from plant species mentioned above.

The method for alleviating or preventing a disease associated with at least one action of the group consisting of OPG, RANKL and osteoclast differentiation inhibition, according to the invention, comprises administrating to a subject a composition containing, as an active ingredient, organic solvent extracts from plant species mentioned above.

The subject to be administered is not particularly restricted and there may be mentioned fish, reptiles, amphibians, feathers, mammals and all animals, and preferred are mammals. The mammals are not particularly restricted and there may be mentioned, for example, humans, monkeys, dogs, cats, bovine species, equine species, swine species, sheep, mice, rats, and guinea pigs, and preferred are humans.

The method for administration is not particularly restricted and there may be mentioned oral, enteric, transdermal and subcutaneous administration, among others, and preferred is oral administration.

In administering the composition mentioned above as a composition for eating or drinking, the dosage is not particularly restricted and that for an adult per day is, for example, 0.01 to 1000 mg/kg body weight, preferably 0.1 to 100 mg/kg body weight, more preferably 1 to 100 mg/kg body weight, in terms of respective extract. Same applies to the case of administering those composition to other animals.

In administering the composition mentioned above as a drug, the dosage is not particularly restricted and that for an adult per day is, for example, 0.01 to 1000 mg/kg body weight, preferably 0.1 to 100 mg/kg body weight, more preferably 1 to 100 mg/kg body weight, in terms of respective extract, and the dosage is administered at one time or several times. Same applies to the case of administering those composition to other animals.

The composition and method of the invention is effective in inducing OPG production or inhibiting RANKL expression in osteoblasts or in inhibiting osteoclast differentiation, among others, thus indirectly or directly inhibiting osteoclast differentiation, so that it can be expected to suppress bone resorption. Therefore, it is useful in alleviating and preventing bone resorption-due diseases, typically osteoporosis. Furthermore, the composition of the invention can be produced from edible materials and can be safely ingested.

### BEST MODES FOR CARRYING OUT THE INVENTION

The following examples illustrate the present invention more specifically. These examples are, however, by no means limitative of the scope of the invention.

### EXAMPLE 1

### Preparation of Extracts from Respective Materials

One part by weight of each plant species was immersed in 5 parts by weight of ethanol and, after 7 days of extraction at room temperature (23° C.), the mixture was filtered to give an extract. The solvent was removed from the extract using an evaporator to give a powder-form extract. The part or region used of each plant and the extractable matter percentage (in % by weight) are shown in Table 1. The extractable matter percentage (in % by weight) refers to the amount of the powder-form extract (in % by weight) relative to 100% by weight of the used material.

TABLE 1

| | | | Extractable |
|---|---|---|---|
| Material | Part or region used | Obtained from | matter (%) |
| Platycodon grandiflorum | Root: dried powder | Kaneka Sun Spice Co., Ltd. | 7.60 |
| Origanum vulgare | Leaves: dried and ground | Kaneka Sun Spice Co., Ltd. | 10.67 |
| Thymus Species | Terrestrial part: dried and ground | Kaneka Sun Spice Co., Ltd. | 14.10 |
| Zanthoxylum bungeanum | Seeds: dried powder | Kaneka Sun Spice Co., Ltd. | 17.24 |
| Zanthoxylum piperitum | Seeds: dried powder | Kaneka Sun Spice Co., Ltd. | 10.50 |
| Arctium lappa | Root: dried and ground | Commercial product | 0.23 |
| Camellia sinensis | Leaves: dried and ground | Commercial product | 1.23 |
| Persea americana fruit | Fruits (skin-free): dried and ground | Commercial product | 50.75 |
| Persea americana fruit skin | Fruit skin: dried and ground | Commercial product | 9.07 |
| Stevia rebaudiana | Leaves: dried and ground | Kaneka Sun Spice Co., Ltd. | 11.26 |
| Thymus serpyllum | Terrestrial part: dried and ground | K-Kobayashi Co. | 6.80 |
| Pimpinella anisum | Fruits: dried and ground | Kaneka Sun Spice Co., Ltd. | 19.46 |
| Matricaria recutita | Flowers: dried and ground | Kaneka Sun Spice Co., Ltd. | 8.91 |
| Malva sylvestris | Leaves/flowers: dried and ground | K-Kobayashi Co. | 4.80 |
| Ilex paraguariensis | Leaves: dried and ground | Commercial product | 8.26 |
| Ptychopetalum olacoides | Bark: dried and ground | Commercial product | 2.06 |

### EXAMPLE 2

### Measurement of OPG Production Promoting Activity in Osteoblasts

The various ethanol extracts obtained in Example 1 were each evaluated for OPG production promoting activity in osteoblasts. Thus, 96-well plates were sowed with 2×10⁴ cells/well of human osteoblasts (MG63 cells), and the cells were cultivated in Eagle's MEM medium (NISSUI PHARMACEUTICAL CO., LTD.) containing 1% of nonessential amino acids (product of Gibco) and 10% of FBS (Fetal Bovine Serum) in an atmosphere of 5% CO₂-95% air at 37° C. for 3 days.

After those 3 days, the medium was replaced with Eagle's MEM containing 1% of nonessential amino acids and 0.125% of bovine serum albumin and, after further 1 day of cultivation in 5% CO₂-95% air at 37° C., the medium was replaced with a 30 or 100 μg/ml solution of each extract obtained in Example 1 in a medium. The medium used for dissolving each sample was Eagle's MEM containing 1% of nonessential amino acids and 0.125% of bovine serum albumin.

Three days after the last medium exchange, the amount of OPG in the culture supernatant was determined by the ELISA technique using an OPG assay kit (Human Osteoprotegerin ELISA; Bio Vendor). The assay was carried out according to the procedure recommended by the manufacturer.

The OPG production promoting activity evaluation was performed using the ratio of the amount of OPG as determined in the case of extract addition to the amount of OPG in the case of solvent control as determined in parallel (OPG production promotion rate), with the mean value for the solvent control being taken as 1. Each assay was repeated three times. The samples giving an OPG production promotion rate of higher than 1 with statistical significance were evaluated as “having OPG production promoting activity”. The extracts for which OPG production promoting activity could be observed are shown in Table 2. Each value is given in terms of mean±standard deviation.

TABLE 2

| | OPG production |
|---|---|
| Sample | promotion rate |
| Platycodon grandiflorum extract (30 μg/ml) | 1.1 ± 0.06 |
| Platycodon grandiflorum extract (100 μg/ml) | 2.1 ± 0.16 |
| Origanum vulgare extract (30 μg/ml) | 2.6 ± 0.25 |
| Origanum vulgare extract (100 μg/ml) | 3.4 ± 0.10 |
| Thymus Species extract (30 μg/ml) | 3.4 ± 0.15 |
| Thymus Species extract (100 μg/ml) | 4.7 ± 0.80 |
| Zanthoxylum bungeanum extract (30 μg/ml) | 2.5 ± 0.22 |
| Zanthoxylum bungeanum extract (100 μg/ml) | 4.0 ± 0.77 |
| Zanthoxylum piperitum extract (30 μg/ml) | 1.6 ± 0.13 |
| Zanthoxylum piperitum extract (100 μg/ml) | 2.1 ± 0.10 |
| Arctium lappa extract (30 μg/ml) | 1.5 ± 0.05 |
| Arctium lappa extract (100 μg/ml) | 3.1 ± 0.43 |
| Camellia sinensis extract (30 μg/ml) | 2.6 ± 0.30 |
| Camellia sinensis extract (100 μg/ml) | 3.0 ± 0.27 |
| Persea americana fruit extract (100 μg/ml) | 1.8 ± 0.03 |
| Persea americana fruit skin extract (100 μg/ml) | 2.3 ± 0.11 |
| Stevia rebaudiana extract (30 μg/ml) | 1.5 ± 0.20 |
| Stevia rebaudiana extract (100 μg/ml) | 2.5 ± 0.24 |
| Thymus serpyllum extract (30 μg/ml) | 2.3 ± 0.05 |
| Thymus serpyllum extract (100 μg/ml) | 3.0 ± 0.27 |
| Pimpinella anisum extract (30 μg/ml) | 1.4 ± 0.12 |
| Pimpinella anisum extract (100 μg/ml) | 2.2 ± 0.08 |
| Matricaria recutita extract (30 μg/ml) | 1.4 ± 0.17 |
| Matricaria recutita extract (100 μg/ml) | 2.1 ± 0.14 |
| Malva sylvestris extract (30 μg/ml) | 1.5 ± 0.24 |
| Malva sylvestris extract (100 μg/ml) | 1.8 ± 0.05 |
| Ilex paraguariensis extract (30 μg/ml) | 1.7 ± 0.21 |
| Ilex paraguariensis extract (100 μg/ml) | 1.3 ± 0.14 |
| Ptychopetalum olacoides extract (30 μg/ml) | 4.2 ± 0.24 |

From the results shown in Table 2, it was confirmed that extracts from plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum show OPG production promoting activity in osteoblasts.

### COMPARATIVE EXAMPLE 1

### Comparison of OPG Production Promoting Activity in Osteoblasts Between Extracts from Plants Belonging to the Genus Platycodon Obtained by Using Different Extraction Solvents

In accordance with Patent Document 2, 1 part by weight of the same Platycodon grandiflorum root powder as used in Example 1 was immersed in 30 parts by weight of distilled water and, after 2 days of extraction at 85° C., an extract was recovered by filtration. A portion of the extract was subjected to ultrafiltration treatment, which gave a fraction with a molecular weight of not higher than 5000. This fraction was lyophilized to give a fraction derived from the water extract from Platycodon grandiflorum with a molecular weight of not higher than 5000 in the form of a dry powder.

Then, the ethanol extract from Platycodon grandiflorum as obtained in Example 1 and the above-mentioned water extract from Platycodon grandiflorum were evaluated for OPG production promoting activity in the same manner as in Example 2. The results are shown in Table 3.

TABLE 3

| | OPG production |
|---|---|
| Sample | promotion rate |
| Ethanol extract from Platycodon grandiflorum | 1.61 ± 0.12* |
| (30 μg/ml) | |
| Ethanol extract from Platycodon grandiflorum | 2.48 ± 0.20* |
| (50 μg/ml) | |
| Ethanol extract from Platycodon grandiflorum | 3.25 ± 0.19* |
| (100 μg/ml) | |
| Water extract from Platycodon grandiflorum | 1.05 ± 0.04 |
| (30 μg/ml) | |
| Water extract from Platycodon grandiflorum | 1.02 ± 0.08 |
| (50 μg/ml) | |
| Water extract from Platycodon grandiflorum | 1.14 ± 0.05 |
| (100 μg/ml) | |
| Fraction (m.w. ≦ 5000) of water extract | 0.85 ± 0.05 |
| from Platycodon grandiflorum (30 μg/ml) | |
| Fraction (m.w. ≦ 5000) of water extract | 0.85 ± 0.03 |
| from Platycodon grandiflorum (50 μg/ml) | |
| Fraction (m.w. ≦ 5000) of water extract | 0.91 ± 0.07 |
| from Platycodon grandiflorum (100 μg/ml) | |

In Table 3, the samples showing data marked with “*” are those samples for which OPG production promoting activity could be established with statistical significance (P<0.01) as statistically analyzed by the Dunnet method. From the results shown in Table 3, it was shown that the OPG production promoting activity-displaying components in Platycodon grandiflorum as described herein are components properly extractable with the organic solvent ethanol, not with water.

### EXAMPLE 3

### Assay of RANKL Expression Inhibiting Activity in Osteoblasts

The various ethanol extracts obtained in Example 1 were each evaluated for RANKL expression inhibiting activity in osteoblasts. Thus, 12-well plates were sowed with 2×10⁵ cells/well of human osteoblasts (MG63 cells), and the cells were cultivated in Eagle's MEM (NISSUI PHARMACEUTICAL CO., LTD.) containing 1% of nonessential amino acids (product of Gibco) and 10% of FBS in an atmosphere of 5% CO₂-95% air at 37° C. for 2 days.

After those 2 days, the medium was replaced with Eagle's MEM containing 1% of nonessential amino acids and 0.125% of bovine serum albumin and, after further 1 day of cultivation in 5% CO₂-95% air at 37° C., the medium was replaced with a 100 μg/ml solution of each extract obtained in Example 1 in a medium. The medium used for dissolving each sample was Eagle's MEM containing 1% of nonessential amino acids and 0.125% of bovine serum albumin.

One day after the last medium exchange, the total RNA in the cells was extracted using an RNA extraction kit (RNeasy Mini kit; Qiagen). Then, using 1 μg of the total RNA obtained, cDNA synthesis was carried out utilizing a cDNA synthesis kit (High Capacity cDNA Archive kit; Applied Biosystems). A one fiftieth portion of the cDNA obtained was subjected to real time PCR, and the levels of expression of the RANKL gene and the GAPDH (glyceraldehyde-3-phosphate dehydrogenase) gene, an internal standard gene, were determined. The enzyme and other reagents as well as the primers and TaqMan probes used in the real time PCR were the products of Applied Biosystems (TaqMan Universal PCR Master Mix, TaqMan Gene Expression Assays). All the procedures were carried out according to the protocols recommended by the manufacturer.

Then, the relative RANKL expression level (measured RANKL value/measured GAPDH value) in each extract treatment was calculated from the thus-obtained measured RANKL and GAPDH values.

The RANKL expression inhibiting activity evaluation was performed using the ratio of the RANKL expression level determined in the case of extract addition treatment to the RANKL expression level in the case of solvent control as determined in parallel (RANKL expression inhibition rate), with the mean value for the solvent control being taken as 1. Each assay was repeated three times. The samples giving a RANKL expression rate of lower than 1 with statistical significance were evaluated as “having RANKL expression inhibiting activity”. The extracts for which RANKL expression inhibiting activity could be observed are shown in Table 4. Each value is given in terms of mean±standard deviation.

TABLE 4

| | RANKL expression |
|---|---|
| Sample | inhibition rate |
| Pimpinella anisum extract (100 μg/ml) | 0.52 ± 0.29 |
| Ilex paraguariensis extract (100 μg/ml) | 0.68 ± 0.24 |
| Platycodon grandiflorum extract (100 μg/ml) | 0.64 ± 0.16 |
| Origanum vulgare extract (100 μg/ml) | 0.11 ± 0.04 |
| Thymus Species extract (100 μg/ml) | 0.09 ± 0.07 |
| Persea americana fruit extract (100 μg/ml) | 0.10 ± 0.04 |
| Thymus serpyllum extract (100 μg/ml) | 0.21 ± 0.06 |

From the results shown in Table 4, it was established that extracts from plants belonging to the genus Platycodon, plants belonging to the genus Origanum, plants belonging to the genus Thymus, plants belonging to the genus Persea, plants belonging to the genus Pimpinella, and plants belonging to the genus Ilex have RANKL expression inhibiting activity in osteoblasts.

### EXAMPLE 4

### Assay of Osteoclast Differentiation Inhibiting Effect in Cocultivation of Osteoblasts and Bone Marrow Cells

48-well plates were sowed with osteoblasts collected from the parietal bone of 1 to 3-day-old DDY mice (1×10⁴ cells/well) and bone marrow cells collected from the femora and tibiae of 5-week-old DDY mice (2×10⁵ cells/well). Simultaneously with the sowing of the cells, the respective ethanol extracts obtained in Example 1 and prostaglandin E2 were added to the cells to an extract concentration of 50 μg/ml and a prostaglandin E2 concentration of 1×10⁻⁶ M. The medium used was A-MEM (Sigma) containing 10% of FBS. After 3 to 4 days from the start of cultivation, the medium in each well was replaced with a fresh portion. Thus, the cell culture was carried out in the presence of each extract and prostaglandin E2 for a total of 7 days. After completion of the cultivation, cells were subjected to formalin fixation and tartrate-resistant acid phosphatase staining (TRAP staining) in the conventional manner. Then, the cells stained by TRAP staining (TRAP-positive cells) were counted under a microscope for osteoclast differentiation evaluation.

The osteoclast differentiation inhibiting activity evaluation was performed using the ratio of the number of TRAP-positive cells as determined in the case of extract addition treatment to the number of TRAP-positive cells in the case of solvent control as determined in parallel (osteoclast differentiation inhibition rate), with the mean value for the solvent control being taken as 1. Each assay was repeated four times. The samples giving an osteoclast differentiation inhibition rate of lower than 1 with statistical significance were evaluated as “having osteoclast differentiation inhibiting activity”. The extracts for which osteoclast differentiation inhibiting activity could be observed are shown in Table 5. Each value is given in terms of mean±standard deviation.

TABLE 5

| | Osteoclast |
|---|---|
| Sample | differentiation |
| Platycodon grandiflorum extract (50 μg/ml) | 0.41 ± 0.03 |
| Persea americana fruit skin extract (50 μg/ml) | 0.28 ± 0.32 |
| Persea americana fruit extract (50 μg/ml) | 0.09 ± 0.06 |
| Stevia rebaudiana extract (50 μg/ml) | 0.11 ± 0.09 |
| Pimpinella anisum extract (50 μg/ml) | 0.39 ± 0.08 |
| Matricaria recutita extract (50 μg/ml) | 0.02 ± 0.04 |
| Malva sylvestris extract (50 μg/ml) | 0.41 ± 0.19 |

From the results shown in Table 5, it was confirmed that extracts from plants belonging to the genus Platycodon, plants belonging to the genus Persea, plants belonging to the genus Stevia, plants belonging to the genus Pimpinella, plants belonging to the genus Matricaria, and plants belonging to the genus Malva have an osteoclast differentiation inhibiting effect.

### EXAMPLE 5

### Confirmation of Effect of Alleviating Symptoms of Osteoporosis Upon Administration to Animals

SD strain female rats 8 weeks of age were fed for 1 week and then osteoporosis was induced by ovariectomy. After further 6 weeks of feeding following ovariectomy, the rats were euthanized, and the femora were excised and subjected to cancellous bone density measurement at the shaft end by the pQCT method (peripheral Quantitative Computed Topography). The feed used was a purified diet according to AIN-93G. However, the calcium content was adjusted to 0.3%. From the day of starting the experiment, the animals were given the feed supplemented with 1.25% by weight of each extract sample, and a group given the feed without admixture of any extract sample was used as a control to confirm an osteoporosis-alleviating effect. The osteoporosis-alleviating effect was expressed in terms of the relative bone density value (mean±standard error) of the extract-dosed group, with the bone density of the control group being taken as 100%. Each group consisted of 8 animals. The extracts subjected to osteoporosis-alleviating effect evaluation were the Pimpinella anisum extract, Stevia rebaudiana extract and Platycodon grandiflorum extract obtained in Example 1.

TABLE 6

| | Cancellous bone |
|---|---|
| Sample | density increase (%) |
| Pimpinella anisum extract-dosed group | 118 ± 3 |
| Stevia rebaudiana extract-dosed group | 114 ± 7 |
| Platycodon grandiflorum extract-dosed group | 110 ± 7 |

Each extract-dosed group was higher in cancellous bone density value as compared with the control group and, thus, it was established that the extracts from plants belonging to the genus Pimpinella, that from plants belonging to the genus Stevia and that from plants belonging to the genus Platycodon have an osteoporosis-alleviating effect. The difference between each extract group and the control group was evaluated for significance by Student's t test. The P value for the extract from plants belonging to the genus Pimpinella was 0.009, the value for that from plants belonging to the genus Stevia was 0.115 and the P value for that from plants belonging to the genus Platycodon was 0.357 and it was thus confirmed that the extract from plants belonging to the genus Pimpinella, in particular, has a high-level osteoporosis-alleviating effect.

### INDUSTRIAL APPLICABILITY

The composition and method of the invention is effective in inducing OPG production or inhibiting RANKL expression in osteoblasts or in inhibiting osteoclast differentiation, among others, thus indirectly or directly inhibiting osteoclast differentiation, so that it can be expected to suppress bone resorption. Therefore, it is useful in alleviating and preventing bone resorption-due diseases, typically osteoporosis. Furthermore, the composition of the invention can be produced from edible materials and can be safely ingested.

1. A method for promoting production of OPG,
which comprises administrating to a subject a composition containing, as an active ingredient, organic solvent extracts from at least one plant species selected from the group consisting of plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum.

2. A method for inhibiting differentiation of osteoclast,
which comprises administrating to a subject a composition containing, as an active ingredient, organic solvent extracts from at least one plant species selected from the group consisting of plants belonging to the genus Platycodon, plants belonging to the genus Persea, plants belonging to the genus Stevia, plants belonging to the genus Pimpinella, plants belonging to the genus Matricaria, and plants belonging to the genus Malva.

3. A method for inhibiting expression of RANKL,
which comprises administrating to a subject a composition containing, as an active ingredient, organic solvent extracts from at least one plant species selected from the group consisting of plants belonging to the genus Platycodon, plants belonging to the genus Origanum, plants belonging to the genus Thymus, plants belonging to the genus Persea, plants belonging to the genus Pimpinella, and plants belonging to the genus Ilex.

4. A method for alleviating or preventing a disease associated with at least one action of the group consisting of OPG, RANKL and osteoclast differentiation inhibition,
which comprises administrating to a subject a composition containing, as an active ingredient, organic solvent extracts from at least one plant species selected from the group consisting of plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum.

5. The method according to claim 4
wherein the disease associated with at least one action of the group consisting of OPG, RANKL and osteoclast differentiation inhibition is osteoporosis.

6. The method according to claim 1
wherein the composition is for eating or drinking.

7. The method according to claim 1 wherein the composition is a medicine.

8. The method according to claim 1 wherein the subject is a mammal.

9. The method according to claim 8
wherein the subject is a human.

10. An OPG production promoter
which contains, as an active ingredient, an organic solvent extract from at least one plant species selected from the group consisting of plants belonging to the genus Platycodon, plants belonging to the genus Origanum, plants belonging to the genus Thymus, plants belonging to the genus Zanthoxylum, plants belonging to the genus Arctium, plants belonging to the genus Camellia, plants belonging to the genus Persea, plants belonging to the genus Stevia, plants belonging to the genus Pimpinella, plants belonging to the genus Matricaria, plants belonging to the genus Malva, plants belonging to the genus Ilex, and plants belonging to the genus Ptychopetalum.

11. An osteoclast differentiation inhibitor
which contains, as an active ingredient, an organic solvent extract from at least one plant species selected from the group consisting of plants belonging to the genus Platycodon, plants belonging to the genus Persea, plants belonging to the genus Stevia, plants belonging to the genus Pimpinella, plants belonging to the genus Matricaria, and plants belonging to the genus Malva.

12. A RANKL expression inhibitor
which contains, as an active ingredient, an organic solvent extract from at least one plant species selected from plants belonging to the genus Platycodon, plants belonging to the genus Origanum, plants belonging to the genus Thymus, plants belonging to the genus Persea, plants belonging to the genus Pimpinella, and plants belonging to the genus Ilex.

13. The method according to claim 2
wherein the composition is for eating or drinking.

14. The method according to claim 3
wherein the composition is for eating or drinking.

15. The method according to claim 4
wherein the composition is for eating or drinking.

16. The method according to claim 2
wherein the composition is a medicine.

17. The method according to claim 3
wherein the composition is a medicine.

18. The method according to claim 4
wherein the composition is a medicine.

19. The method according to claim 2
wherein the subject is a mammal.

20. The method according to claim 3
wherein the subject is a mammal.
